(19) United States
(12) Patent Application Publication
(10) Pub. No.: US 2015/0150898 A1
(43) Pub. Date: Jun. 4, 2015

(54) NUTRITIONAL PRODUCTS AND METHODS OF IMPROVING EXERCISE PERFORMANCE AND REDUCING EXERCISE INDUCED IMPAIRMENT OF IMMUNE FUNCTION

(71) Applicant: NU SCIENCE LABORATORIES, INC., Chestnut Hill, MA (US)

(72) Inventor: Mark G. Connell, Chestnut Hill, MA (US)

(21) Appl. No.: 14/406,752

(22) PCT Filed: Jun. 11, 2013
(86) PCT No.: PCT/US13/45202

§ 371 Date: Dec. 10, 2014

Related U.S. Application Data
(60) Provisional application No. 61/658,142, filed on Jun. 11, 2012.

(51) Int. Cl. A61K 31/7088; A61K 38/02; A23L 1/29; A61K 36/064; A61K 36/06; A61K 31/70; A61K 31/202

(52) U.S. Cl. CPC A61K 31/7088; A61K 31/70; A61K 38/02; A61K 31/202; A61K 36/064; A61K 36/06; A23L 1/293; A23V 2002/00

(57) ABSTRACT

Nutritional products comprising a nucleotide component and optionally, a carbohydrate component, a protein component and a polyunsaturated fatty acid component are disclosed. The products can be in various product forms, including solid and liquid forms, such as drinks, bars, and gels. Also disclosed are methods for inhibiting exercise-induced impairment of immune system functioning and for improving exercise performance by administration of compositions containing nucleotides.

This application claims priority to U.S. Provisional Application No. 61/658,142 for “Nutritional Products and Methods of Diet Supplementation” filed on Jun. 11, 2012, which is hereby incorporated by reference in its entirety.

### FIELD OF THE INVENTION

The present invention is directed to nutritional products, including solid and liquid products, useful for athletes during recovery from exercise and methods of use of the same.

### BACKGROUND OF THE INVENTION

Nutritional products for athletes typically address a single need of the athlete. Most products focus on supply of either energy (carbohydrate and electrolyte based products) or protein. When taken within an hour after exercise, carbohydrates help to rebuild muscle and liver glycogen. Protein and amino acid based products can help speed absorption of carbohydrates and provide building blocks to repair muscles.

None of these types of products can address the initiation of tissue recovery, which is mediated by the immune system. In addition, there is a frequently noted correlation between prolonged heavy exercise and upper respiratory tract infection (URTI). While the causal pathways are not certain, it is recognized that exercise has multiple effects on the immune system which might explain these observations.

It is widely accepted that both acute and chronic exercise alter the number and function of circulating cells of the innate immune system (eg, neutrophils, monocytes and natural killer (NK) cells). Similarly, it is agreed that a lymphocytosis is observed during and immediately after exercise, proportional to exercise intensity and duration, with the number of cells (T cells and to a lesser extent B cells) falling below pre-exercise levels during the early stages of recovery, before returning to resting values normally within 24 hours. Finally, a consensus exists that reduced levels of secretory immunoglobulin A (SIgA) are associated with increased risk for URTI during heavy training as the production of SIgA is the major effector function of the mucosal immune system providing the ‘first line of defense’ against pathogens. Thus, there is an ongoing need to manage and reduce the negative effects of exercise on the immune system to keep athletes healthy and performing at their highest levels.

Further, independent of immune system effects, there is a need to improve performance levels of athletes for athletes of all calibers.

### SUMMARY OF THE INVENTION

One embodiment of the present invention is a sports nutritional product that includes a nucleotide component, a carbohydrate component, a protein component; and a polyunsaturated fatty acid (PUFA) component. This product is multi-functional and provides energy and protein, reduce inflammation resulting from exercise and initiates tissue recovery from exercise-induced damage.

The product can be a liquid product or a solid product and if it is a liquid can be drinkable and be either a clear liquid product and shake-like product. The liquid product can include electrolytes, such as at a concentration between about 20 mmol/liter and about 40 mmol/liter. If the product is a solid product, it can be selected from bars, cookies, gels, powders and capsules.

The nucleotide component of the product can include RNA and DNA and can include an extract of a yeast, such as a yeast selected from the group consisting of Saccharomyces cerevisiae and Candida utilis. The nucleotide component of the nutritional product can be present in an amount between about 10 mg and about 1000 mg per serving, between about 100 mg and about 900 mg per serving, or. between about 400 mg and about 800 mg per serving.

The carbohydrate component of the nutritional product can be present in an amount between about 2 gm and about 40 gm per serving, between about 10 gm and about 100 gm per serving, or between about 50 mg and about 4000 mg per serving. Further the carbohydrate component can be selected from glucose, sucrose, fructose, dextrose, lactose and combinations thereof.

The protein component can be selected from soy protein, whey protein, casein protein and combinations thereof.

The PUFA component of the nutritional component can be an ω-3 PUFA, which can be selected from docosahexaenoic acid, omega-3 docosapentaenoic acid, eicosapentaenoic acid, stearidonic acid, α-linolenic acid, and mixtures thereof or from docosahexaenoic acid, ω-3 docosapentaenoic acid, eicosapentaenoic acid and mixtures thereof.

A further embodiment of the present invention is a method to inhibit exercise-induced impairment of immune function by administering a composition to an athlete engaging in exercise, wherein the composition comprises a nucleotide component. The composition can further include a component selected from a carbohydrate component, a protein component, an ω-3 fatty acid component; and combinations thereof and can be selected from specific nutritional products described herein. In the method, the composition can be administered prior to the exercise, such as for at least about 1 week, 2 weeks or more. Also, the composition can be administered after the exercise, such as for at least about 1 week, 2 weeks or more.

The method is suitable for any athlete or individual engaging in athletic endeavors. More particularly the athlete can be an endurance athlete or a strength athlete and can be male or female. The method is particularly suitable for athletes 20 years or older, 30 years or older, 40 years or older, 50 years or older, or 60 years or older.

The type of exercise can be resistance exercise, including acute heavy resistance exercise. Such exercise can be weightlifting, sprinting, field events, football, martial arts, wrestling, or boxing. The exercise can also be endurance exercise, such as running, cycling, swimming, hiking, triathlon, softball, baseball, soccer, basketball, hockey, football, rugby, tennis, and lacrosse.

A still further embodiment of the present invention is a method to improve exercise performance by administering a composition to an athlete engaging in exercise, wherein the composition comprises a nucleotide component. The composition can further include a component selected from a carbohydrate component, a protein component, an ω-3 fatty acid component; and combinations thereof and can be selected from specific nutritional products described herein. In the method, the composition can be administered prior to the exercise, such as for at least about 1 week, 2 weeks or more. Also, the composition can be administered after the exercise, such as for at least about 1 week, 2 weeks or more.

The method is suitable for any athlete or individual engaging in athletic endeavors. More particularly the athlete can be an endurance athlete or a strength athlete and can be male or female. The method is particularly suitable for athletes 20 years or older, 30 years or older, 40 years or older, 50 years or older, or 60 years or older.

The type of exercise can be resistance exercise, including acute heavy resistance exercise. Such exercise can be weightlifting, sprinting, field events, football, martial arts, wrestling, or boxing. The exercise can also be endurance exercise, such as running, cycling, swimming, hiking, triathlon, softball, baseball, soccer, basketball, hockey, football, rugby, tennis, and lacrosse.

### BRIEF DESCRIPTION OF THE FIGURES

FIG. 1 illustrates an acute heavy resistance exercise test protocol for compositions of the invention.

FIG. 2 illustrates a testing timeline for evaluating athletes in the test protocol of FIG. 1.

### DETAILED DESCRIPTION

Various embodiments of the present invention include products that comprise a nucleotide component and one or more additional components, as well as related methods of administration, such as for treating athletes. Such additional components can include, for example, a carbohydrate component, a protein component and/or a polyunsaturated fatty acid (PUFA), among other components described herein.

One embodiment of the present invention is a sports nutritional product that includes a nucleotide component, a carbohydrate component, a protein component, and a polyunsaturated fatty acid (PUFA) component. Such products are primarily intended for use by athletes and individuals involved in athletic training. Therefore, such individuals are adults and teenagers who eat a diet of solid food. Such individuals do not include infants or babies, and therefore, sports nutritional products of the present invention do not include infant formulae or other types of food intended to be fed to infants or babies. Accordingly, sports nutritional products of the present invention do not include ingredients that are unique to infant formulae or other types of food intended to be fed to infants or babies. For example, such ingredients in infant formulae or other types of food intended to be fed to infants or babies but not in sports nutritional products of the present invention can be arachidonic acid. Sports nutritional products of the present invention including nucleotide, carbohydrate, protein and PUFA components provide a unique combination of nutritional elements for an athlete.

There are numerous existing products that provide carbohydrates as well as products that provide protein. Many offer them in various combinations. Carbohydrates provide the raw material to quickly rebuild the body's readily available energy stores (ie, glycogen stored in both muscles and the liver), particularly if consumed within a short time period after exercise when the hormonal environment in the body allows rapid conversion of glucose to glycogen. Protein (particularly those high in branched chain amino acids) provides raw material for rebuilding muscles. However, neither of these components, alone or in combination, provides the immune system benefits supported by the availability of nucleotides in compositions of the present invention.

### Nucleotide

The term nucleotide refers to a compound that has a nitrogenous base, a pentose sugar and one or more phosphate groups. The nitrogenous base is either a purine or a pyrimidine. Pyrimidine bases are six-membered rings, and include uracil (U), cytosine (C) and thymine (T). Purine bases have a second five-membered ring, and include adenine (A), guanine (G), hypoxanthine and xanthine. A purine or pyrimidine based linked to a pentose molecule constitutes a nucleoside. A nucleotide is a phosphate ester of a NS, and may occur in the monophosphate, diphosphate or triphosphate form. The pentose is either ribose or deoxyribose; the ribonucleotide and deoxynucleotide serve as the monomeric units of RNA and DNA, respectively. RNA and DNA are linear polymers consisting of four different nucleotides linked together by 5′,3′ phosphodiester bonds. Nucleotides of the present invention include any compound or composition that is a source of nucleotides, and thus, can include individual nucleotides, dinucleotides, trinucleotides and oligonucleotides (comprising up to about 50 bases), as well as longer polynucleotides.

Nucleotide components of the present invention can comprise extracts of yeast, such as Saccharomyces cerevisiae (brewer's yeast) or Candida utilis (formerly Torulopsis utilis or Torula utilis) (produced from wood sugars as a by-product of paper production).

Suitable nucleotide sources for the present invention include commercially available ones such as nuBound Recovery Supplement (Nu Science Laboratories, Inc., Chestnut Hill, Mass.). Other commercially available products include Life Extension RNA Capsules; Bluebonnet Kosher Nucleotide Complex 300; Good N Natural RNA/DNA; VitaminLife DNA/RNA; and Country Life RNA/DNA.

Nucleotide components of the present invention can be present in products such that the amount of nucleotide delivered per serving is between about 10 mg and about 1000 mg, between about 100 mg and about 900 mg, or between about 400 mg and about 800 mg. In other embodiments, the nucleotide component per serving can be present in ranges having a lower end of the range of about 10 mg, about 20 mg, about 30 mg, about 40 mg, about 50 mg, about 60 mg, about 70 mg, about 80 mg, about 90 mg, about 100 mg, about 200 mg, about 300 mg, or about 400 mg and having a higher end of the range of about 1000 mg, about 900 mg, about 800 mg, about 700 mg, about 600 mg, about 500 mg, about 400 mg, about 300 mg, about 280 mg, about 260 mg, about 240 mg, about 220 mg, about 200 mg, about 190 mg, about 180 mg, about 170 mg, about 160 mg, about 150 mg, about 140 mg, about 130 mg, about 120 mg, about 110 mg, or about 100 mg. It should be recognized that the foregoing amounts are per serving and that a serving can be prepared in different product forms depending on how much of a nucleotide component is included in a product. For example, a serving may be one-half or an entire sports drink, an entire sports bar, two cookies or 4 capsules.

Without intending to be bound by theory, in various products of the present invention, the nucleotide component can play one or more of a variety of functions. Nucleotides are a key component in several major processes within the body and play key roles in many biological processes. The requirements for nucleotides may increase when recovering from major tissue injury, systemic infection or possible when liver function in suppressed. A nucleotide rich diet has been shown to result in improved immune function, improved growth and resistance to infection. Nucleotides can offset the negative hormonal response associated with metabolic or inflammatory insult, such as by demanding endurance exercise (i.e., exercise using slow twitch muscle fibers and aerobic energy pathways) and/or demanding strength and power exercise (i.e., exercise using fast twitch muscle fibers and anaerobic energy pathways).

Additional components of products of the present invention, include carbohydrates, proteins (including protein derivatives, such as hydrolysates and amino acids), polyunsaturated fatty acids (particularly, including ω-3 polyunsaturated fatty acids), antioxidants (e.g., vitamins C and E, beta-carotene, N-acetylcysteine and butylated hydroxyanisole), glutamine, ginseng, echinacea, bovine colostrum, immunoferon, zinc, plant sterols, and non-steroidal anti-inflammatory compounds.

### Carbohydrates

As used herein, “carbohydrate” refers to a source of carbohydrates such as, but not limited to, monosaccharides, disaccharides, oligosaccharides, polysaccharides or derivatives thereof. Such carbohydrates can include without limitation glucose, sucrose, fructose, dextrose and lactose. Particularly preferred sugars include glucose, dextrose, lactose and other non-fructose-based saccharides. Another preferred carbohydrate is a large glucose polymer having a molecular weight between 500,000 and 700,000 which is known commercially as SuperStarch. This product has a low osmolality with a slow “time-released” glucose profile and low insulin impact to avoid the spike and crash phenomena and extend maintenance of blood glucose. This product is available from the UCAN Company of Woodbridge, Conn.

Carbohydrate ingestion is known to stimulate the secretion of insulin which in turn facilitates the uptake of glucose into skeletal muscle via glucose transporter 4 (GLUT4) translocation. Glucose is then converted to and stored as glycogen and triglycerides. Concomitant with this, insulin inhibits the release and synthesis of glucose. Moreover, insulin also plays an important role in protein metabolism where it inhibits the breakdown of protein or proteolysis. Furthermore, insulin promotes the uptake of amino acids into muscle and stimulates protein synthesis, particularly following exercise. Insulin has also been shown to stimulate creatine uptake by muscle cells. Alpha Lipoic Acid has been shown to have insulin-like properties, which further aid in the retention of Creatine. Via the combination of creatine with alpha lipoic acid and a small amount of carbohydrate, creatine retention as compared to Creatine and carbohydrate alone is significantly improved. Therefore, it is advantageous that, for the purposes of creatine retention, the actions of insulin be enhanced. Furthermore, it may be advantageous to increase the activity and availability of creatine in skeletal muscle. Carbohydrates have been shown to attenuate increases in plasma cytokines and stress hormones during demanding endurance exercise.

Some embodiments of the present invention include liquid drink products. While rehydration can be a major reason to consume drink products, the ability to furnish rapidly-absorbed carbohydrates can be very important. Hundreds of research studies over the past 3 decades have proven that ingesting carbohydrates during prolonged strenuous exercise enhances performance compared to plain water (Hawley J A, et al. 1992). Thus, drink products can serve two roles: (a) rehydration and (b) fuel for exercising muscles. Research shows that, when the carbohydrate concentration in a beverage increases above 8%, gastric emptying slows (Mitchell J B, et al. 1989) (Murray R, et al. 1999). This is a critical factor in the development of drink products since strenuous exercise by itself can impair gastric emptying: as exercise intensity increases, the rate of gastric emptying decreases (Moodley D, et al. 1992; Leiper J B, et al. 2001). Thus, if one drinks beverages with a carbohydrate concentration greater than 8% while running, not only does this impair the delivery of fuel to exercising muscles, you may be more likely to develop gastrointestinal intolerance (stomach cramps, vomiting).

According to some research, the optimum concentration of carbohydrate in a drink product is 6-8%. This concentration can also be expressed as:
6-8 grams per 100 ml
14.2-18.9 grams per 8 oz.
21.3-28.4 grams per 12 oz.

Further, it has been shown that absorption and oxidation of carbohydrates during strenuous exercise is optimized if carbohydrates are supplied in a mixture of several types, as opposed to a single carbohydrate source (Jentjens R L, et al. 2004a; 2004b). For example, carbohydrates can come from a mixture of sucrose syrup and glucose-fructose syrup.

More generally in products of the present invention, the amount of carbohydrates can be present in products such that the amount of carbohydrate delivered per serving is between about 2 gm and about 40 gm, between about 5 gm and about 30 gm, or between about 10 gm and about 20 gm. In other embodiments, the carbohydrate component per serving can be present in ranges having a lower end of the range of about 2 gm, about 3 gm, about 4 gm, about 5 gm, about 6 gm, about 7 gm, about 8 gm, about 9 gm, about 10 gm and having a higher end of the range of about 40 gm, about 38 gm, about 36 gm, about 34 gm, about 32 gm, about 30 gm, about 28 gm, about 26 gm, about 24 gm, about 20 gm, about 18 gm, about 16 gm, about 14 gm, about 12 gm, or about 10 gm.

### Polyunsaturated Fatty Acids (PUFA)

In some embodiments of the present invention, nutrition products include a PUFA, which can be a PUFA having a chain length of at least 18 carbons or at least about 20 carbons. PUFAs useful in the present invention can be ω-3 (final double bond three positions from the methyl end of the fatty acid). In some embodiments, the PUFA has at least three double bonds. Examples of PUFAs are docosahexaenoic acid C22:6(n-3) (DHA), omega-3 docosapentaenoic acid C22:5(n-3) (DPA), eicosapentaenoic acid C20:5(n-3) (EPA), stearidonic acid, α-linolenic acid (ALA) or mixtures thereof. The PUFAs can be in any of the common forms found in natural lipids including but not limited to triacylglycerols, diacylglycerols, monoacylglycerols, phospholipids, free fatty acids, esterified fatty acids, or in natural or synthetic derivative forms of these fatty acids (e.g. calcium salts of fatty acids, ethyl esters, etc). Reference to a PUFA-containing composition, as used in the present invention, can refer to either a composition comprising only a single PUFA such as DHA or a composition comprising a mixture of two or more PUFAs such as DHA and EPA, DHA and DPA, EPA and DPA or DHA, DPA, and EPA.

In some embodiments, the PUFA-containing composition is selected from the group of a microbial oil, a plant seed oil, and an aquatic animal (e.g., fish) oil. More generally in products of the present invention, the amount of PUFA can be present in products such that the amount of PUFA delivered per serving is between about 50 mg and about 4000 mg, between about 100 mg and about 3000 mg, between about 150 mg and about 2000 mg, or between about 200 mg and about 1000 mg. In other embodiments, the PUFA component per serving can be present in ranges having a lower end of the range of about 50 mg, about 60 mg, about 70 mg, about 80 mg, about 90 mg, about 100 mg, about 125 mg, about 150 mg, about 175 mg, about 200 mg, about 250 mg, about 300 mg, about 350 mg, about 400 mg, about 450 mg, about 500 mg, about 600 mg, about 700 mg, about 800 mg, about 900 mg or about 1000 mg and having a higher end of the range of about 4000 mg, about 3500 mg, about 3000 mg, about 2500 mg, about 2000 mg, about 1500 mg, about 1400 mg, about 1300 mg, about 1200 mg, about 1100 mg, about 1000 mg, about 900 mg, about 800 mg, about 700 mg, about 600 mg, or about 500 mg.

PUFAs of compositions of the present invention can provide an important anti-inflammatory activity to inhibit inflammation occurring as a result of athletic endeavors. It has been recognized that prolonged aerobic (or endurance) exercise as well as strength or resistance exercise provide inflammatory stimuli occurring as a result of muscle damage from the exercise. It is thought that the primary functions of exercise-induced leukocytes are attack and breakdown of debris, clearance of cellular debris and regeneration of muscle cells. The immune response to endurance and resistance exercise serve the same purpose and have similarities in leukocytotic patterns, resistance exercise-induced leukocytosis is less pronounced with the exception that lymphocyte response seems to be increased dramatically immediately post-resistance exercise. Kelly, Neil A. Jr., “The Effect of Total Work Performed During Acute Heavy Resistance Exercise on Circulating Lymphocytes in Untrained Men” (2011). Master's Theses. Paper 48. http://digitalcommons.uconn.edu/gs_theses/48.

### Proteins

Sports nutrition products that contain protein components can be more efficient at increasing both muscle glycogen and the muscle protein after strenuous exercise. Several studies show that exercise performance is better in athletes who consume carbohydrate and protein-containing beverages compared to athletes who received the carbohydrate-only type. Further, muscle injury and/or soreness may be less when protein is ingested after strenuous exercise. Several studies show that CPK levels (a marker for muscle injury) are lower if a carbohydrate and protein-containing product is consumed immediately following exercise. Marine recruits who received protein supplementation immediately postexercise had less muscle soreness.

Protein for products of the present invention can be derived from a variety of sources. For example, soy protein, whey protein or casein protein can be used. Protein sources can be hydrolyzed to varying degrees, including smaller protein molecules, peptides (including dipeptides, tripeptides and oligopeptides) and single amino acids. Preferred sources of protein are sources that are relatively high in the content of branched chain amino acids (leucine, isoleucine and valine), such as whey protein.

Preferred single amino acid sources include the amino acid arginine. In embodiments including arginine, it is particularly preferred to include ω-3 PUFA because of their anti-inflammatory properties.

Protein components of the present invention are present in products such that the amount of protein delivered per serving is between about 10 gm and about 100 gm, between about 20 gm and about 80 gm, or between about 30 gm and about 60 gm. In other embodiments, the nucleotide component per serving can be present in ranges having a lower end of the range of about 10 gm, about 20 gm, about 30 gm, about 40 gm, or about 50 gm, and having a higher end of the range of about 100 gm, about 90 gm, about 80 gm, about 70 gm, about 60 gm, or about 50 gm.

### Antioxidants

Various embodiments of the present invention include antioxidants, including but not limited to vitamin C, vitamin E, beta-carotene, lutein, lycopene, Vitamin B2, CoEnzyme Q10, cysteine, N-acetylcysteine, butylated hydroxyanisole, manganese, copper, zinc and selenium. Antioxidants can be used in products of the present invention in amounts known to those of skill in the art.

### Non-Steroidal Anti-Inflammatory (NSAID)

Various embodiments of the present invention include the use of non-steroidal anti-inflammatory (NSAID) compounds. Such components can offset the negative hormonal response associated with metabolic or inflammatory insult, such as by demanding endurance exercise. NSAIDs useful in the present invention can include without limitation, salicylates (aspirin (acetylsalicylic acid), diflunisal, salsalate); propionic acid derivatives (ibuprofen, Naproxen, Fenoprofen, Ketoprofen, Flurbiprofen, Oxaprozin, Loxoprofen); acetic acid derivatives; enolic acid (Oxicam) derivatives; fenamic acid derivatives (Fenamates); selective COX-2 inhibitors (Coxibs); and sulphonanilides.

Products of the present invention can be in various forms. For example, the products can be either liquid products that are drinkable or they can be solid forms that can be eaten.

Various embodiments of the present invention include drinkable liquids including one or more of the active components described above. Drinkable liquids include clear liquid products as well as shake-like products. Drinkable liquids include a fluid, typically water, and other components. In addition to other components described herein, drinkable liquids can include electrolytes, such as sodium, potassium and other known electrolytes. The optimum concentration of sodium in a sports rehydration drink is 20-40 mmol/liter.

In drinkable liquids that include carbohydrates, such carbohydrates can include, for example, fructose, glucose, maltodextrin, sucrose or combinations thereof. Such drinkable liquids can include carbohydrates in a concentration of about 2.0-8.75% with an osmolality of less than about 400 mOsm.

Solid product forms of the sports nutritional products can include, without limitation bars, cookies, gels, powders, capsules.

A preferred embodiment of the present invention is a composition comprising a nucleotide component, a carbohydrate component, a protein component and PUFA component. This composition is particularly useful for aiding in the recovery of athletes after a workout. The functions of the various components are described in detail above. The composition can be formulated in a number of different product types. Preferred formulations include a drinkable liquid, for example, as a shake-type drink. Alternatively, this composition can be formulated as a dry powder that is rehydrated with water to form a drinkable product. Other embodiments include forming the composition in an edible form such as a bar, cookie or gel.

Products of the present invention can include other components useful for preparing the products in conventional forms such as shakes, drinks, bars, etc. For example, such other components can include flavors, sweeteners, colors, binders, texture modifiers and the like.

Another embodiment of the present invention is a method to inhibit exercise-induced impairment of immune function by administering a composition to an athlete engaging in exercise, wherein the composition includes a nucleotide component. As discussed above, there is a correlation between exercise and reduced immune function. The present invention, involving the administration of nucleotide-containing compositions to athletes and those involved in athletic pursuits, can reduce or inhibit the suppression of immune function associated with exercise in the absence of administration of compositions of the present invention. As such, the present invention can improve the health of athletes. This method is particularly useful for athletes, who in addition to the risk of exercise-induced impairment of immune function, otherwise have or at risk of having impaired immune function. Such additional risk factors can include, without limitation, level of stress, poor nutrition, and lack of sufficient sleep.

For example, immune function can be measured in a variety of ways known to those skilled in the art. For examples, body samples, such as blood samples, from an athlete can be analyzed for blood cell counts, such as by conducting a complete blood count (CBC) and/or looking at one or more markers. For example, samples can be evaluated for hormones or markers thereof (e.g., catecholamines, cortisol or lactate, wherein for example, reduced levels of cortisol after exercise indicate improved function), nucleotide markers (e.g., glutamine, glutamate, or uric acid wherein increased levels indicate use of nucleotides by the body), or markers of oxidative stress (e.g., malondialdehyde (MDA) or myeloperoxidase (MPO) wherein reduced levels indicate improved function). Also, samples can be evaluated for levels of SIgA and/or natural killer (NK) cells, wherein for example, increased levels of either or both indicates improved function.

A further method of the present invention is a method to improve exercise performance by administering a composition to an athlete engaging in exercise, wherein the composition comprises a nucleotide component. There is always a need for athletes, whether or not competitive athletes, to improve their performance in their selected athletic pursuit. The present invention, involving the administration of nucleotide-containing compositions to athletes and those involved in athletic pursuits, can improve the performance of the athlete in their sport or athletic endeavor as compared to performance of the athlete in the absence of administration of compositions of the present invention.

Quantification of improved performance will of course depend on the nature of the athletic endeavor involved. For example, in the instance of a weightlifter, improved performance can be easily objectively measured for example, by the ability to perform a weightlifting exercise at either greater weights or higher repetitions. Similarly, in the case of a runner, improved performance can be measured by completing a fixed distance in a shorter time period.

In preferred embodiments of methods of the invention, such individuals are athletes that have physically exerted themselves through exercise and/or are otherwise susceptible to muscle injury, inflammation and other conditions associated with physical exertion. Such athletes are typically human athletes but can also be non-human athletes such as racehorses and racing dogs. The methods include administering a nutritional product to the athlete before and/or after the athlete has physically exerted himself/herself. The nutritional product includes a nucleotide component and optionally, can include a component selected from a carbohydrate component, a protein component, a PUFA component and combinations thereof. The various embodiments of the product components are described above.

Athletes for whom the present invention is useful can be male or female and in various embodiments, can be 20 years or older, can be 30 years or older, can be 40 years or older, can be 50 years or older, can be 60 years or older. Such athletes can be characterized, for example, as endurance athletes or as strength athletes, although a given individual can of course be in both categories. An endurance athlete, for example, typically participates in sports, exercise or similar endeavors involving the use and development of cardiovascular capability, such as long distance running, cycling, swimming, hiking, triathlon, softball, baseball, soccer, basketball, hockey, football, rugby, tennis, lacrosse and so forth. Such endurance endeavors typically involve exercise using slow twitch muscle fibers and aerobic energy pathways. A strength athlete, for example, typically participates in sports, exercise or similar endeavors involving the use and development of strength capability, such as weightlifting, sprinting, field events (e.g., shot put), football, martial arts, wrestling, boxing, etc. Such strength endeavors typically involve exercise using fast twitch muscle fibers and anaerobic energy pathways. Other relevant sports include archery, basketball, badminton, volleyball, canoeing, diving, fencing, gymnastics, handball, hockey, rowing, sailing, softball, cricket, field hockey, skateboarding, snowboarding, surfing, bowling, golf, rockclimbing, mountaineering, raquetball, squash, skiing, and skating.

Sports exist on a continuum that runs from pure strength-power sports that are short duration/high intensity (eg, weightlifting, shotput, hammer throw, etc) to those that are pure endurance sports that are long duration/low intensity (eg, marathon running, ultra-marathons, ironman triathlons, etc). On the strength-power end of the spectrum athletes are using predominantly fast twitch muscle fibers, which are best trained with resistance exercise, while on the endurance end of the spectrum athletes are using predominantly slow twitch muscle fibers, which are best trained with endurance exercise. Anaerobic metabolism predominates in strength-power events, while aerobic metabolism predominates in endurance sports.

The majority of sports exist along the middle of this spectrum and require bursts of effort at times followed by opportunities for recovery. As such, athletes will typically combine resistance and endurance exercises in their training to produce a balance of strength and endurance. For example, a sprinter would focus on short repeats and incorporate a relatively large component of strength (resistance) training, but would still include some longer runs in their training program. On the other hand a marathon runner would train with runs that might last several hours, including a certain number of long repeats and would minimize resistance training.

Products of the present invention can be administered before and/or after an individual has engaged in a particular form of exercise and physically exerted himself or herself. Such physical exertion is typically the participation in one or more sports described above, such as running, cycling or weightlifting. One method of measuring physical exertion is by the heart rate of the individual. For example, the product can be administered after an individual has achieved an elevated heart rate, and more particularly, achieved an elevated heart rate for at least about 15 minutes, at least about 30 minutes, at least about 45 minutes, at least about 1 hour, at least about 1.5 hours, at least about 2 hours, at least about 2.5 hours, or at least about 3 hours. In this context, an elevated heart rate can be an average heart rate during the physical exertion of at least about 80 beats per minute (BPM), at least about 90 BPM, at least about 100 BPM, at least about 110 BPM, at least about 120 BPM, at least about 130 BPM, at least about 140 BPM or at least about 150 BPM.

More particularly, the product can be administered either before, during and/or after the physical exertion. For example, the product can be administered on a chronic basis, such as daily, every other day, every third day, etc. for a period of time of at least about 1 week, at least about 2 weeks, at least about 3 weeks, at least about 4 weeks, at least about 2 months, at least about 3 months at least about 6 months or longer.

### Example

The following example is a study demonstrating that methods of the invention involving the administration of a nucleotide-containing composition to athletes can improve immune function and athletic performance.

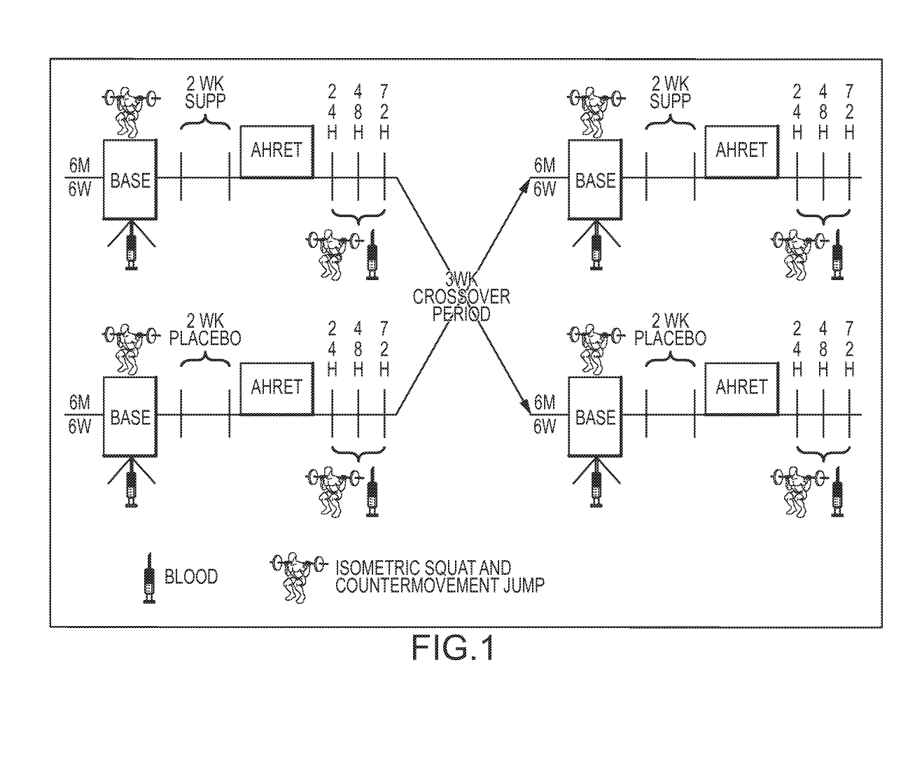

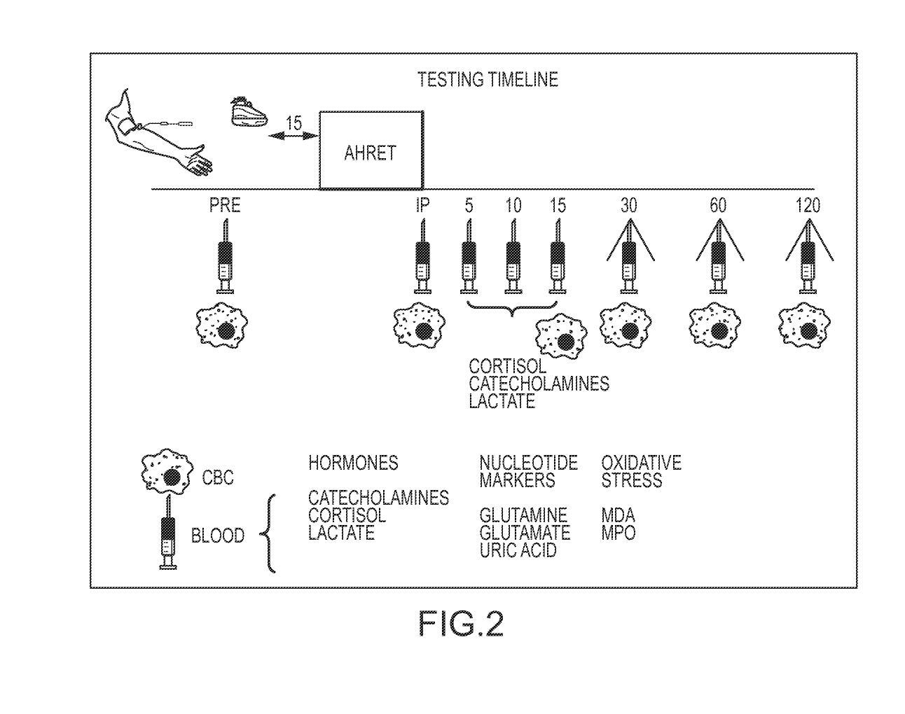

The study design is illustrated in FIGS. 1 and 2. With reference to FIG. 1, the study includes a total of twenty four participants (twelve men and twelve women) who crossover in the middle of the study from treatment to placebo groups (on a double blind basis) such that each subject functions as their own control.

The athletes are tested at the beginning of the study using blood draws, isometric squats and countermovement jumps to establish a baseline (FIG. 1). After baseline evaluation, one group received 2 weeks of nucleotide-containing supplement (nuBound Recovery Supplement (Nu Science Laboratories, Inc., Chestnut Hill, Mass.)) of four capsules/day (approximately 556 mg nucleotides/day) and the other group received placebo. Following this treatment the athletes perform an Acute Heavy Resistance Exercise Test (AHRET) which comprises six sets of ten repetitions of a squat using a weight 60% of the athlete's one rep maximum. Blood draws are taken prior to the AHRET, immediately upon completion and then at 5, 10, 15, 30, 60 and 120 minutes following the work (FIG. 2). Additional blood draws, isometric squats and countermovement jumps are done at 24, 48 and 72 hours following the work. These twelve measurements of blood and the associated workload and force measurements provide a detailed time series following the recovery process, highlighting the immune system changes and capturing changes in athletic performance. The blood draws are analyzed for blood cell counts and hormones or markers thereof (i.e., catecholamines, cortisol and lactate), nucleotide markers (i.e., glutamine, glutamate, and uric acid), and markers of oxidative stress (i.e., malondialdehyde (MDA) or myeloperoxidase (MPO)).

Following the first phase of the study there is a three week washout period where the athletes are subject to no treatment allowing their bodies to return to baseline levels. At that point the entire treatment/testing process is repeated with the athletes receiving whatever treatment (whether nuBound or placebo) that they did not receive in the first phase.

The results will show a statistically significant increase in athletic performance by the athletes in the test groups as compared to the placebo groups. Further, the results will show a statistically significant inhibition of exercise-induced impairment of immune function and lower levels of oxidative stress in the athletes in the test groups as compared to the placebo groups.

While various embodiments of the present invention have been described in detail, it is apparent that modifications and adaptations of those embodiments will occur to those skilled in the art. However, it is to be expressly understood that such modifications and adaptations are within the spirit and scope of the present invention.

What is claimed is:

1. A sports nutritional product, comprising:
a. a nucleotide component;
b. a carbohydrate component;
c. a protein component; and
d. a polyunsaturated fatty acid (PUFA) component.

2. The nutritional product of claim 1, wherein the product is a liquid product.

3-4. (canceled)

5. The nutritional product of claim 2, wherein the liquid product further comprises electrolytes.

6-9. (canceled)

10. The nutritional product of claim 1, wherein the nucleotide component comprises an extract of a yeast.

11. (canceled)

12. The nutritional product of claim 1, wherein the nucleotide component is present in an amount between about 10 mg and about 1000 mg per serving.

13-14. (canceled)

15. The nutritional product of claim 1, wherein the carbohydrate component is present in an amount between about 2 gm and about 40 gm per serving.

16. The nutritional product of claim 1, wherein the protein component is present in an amount between about 10 gm and about 100 gm per serving.

17. The nutritional product of claim 1, wherein the PUFA component is present in an amount between about 50 mg and about 4000 mg per serving.

18-19. (canceled)

20. The nutritional product of claim 1, wherein the PUFA component comprises an ω-3 PUFA.

21-22. (canceled)

23. A method to inhibit exercise-induced impairment of immune function, comprising administering a composition to an athlete engaging in exercise, wherein the composition comprises a nucleotide component.

24. The method of claim 23, wherein the composition further comprises a component selected from the group consisting of:
i. a carbohydrate component;
ii. a protein component;
iii. an ω-3 fatty acid component; and
iv. combinations thereof.

25-31. (canceled)

32. The method of claim 23, wherein the nucleotide component comprises an extract of a yeast.

33. (canceled)

34. The method of claim 23, wherein the nucleotide component is present in an amount between about 10 mg and about 1000 mg.

35-36. (canceled)

37. The method of claim 23, wherein the athlete is an endurance athlete.

38. The method of claim 23, wherein the athlete is a strength athlete.

39-50. (canceled)

51. A method to improve exercise performance, comprising administering a composition to an athlete engaging in exercise, wherein the composition comprises a nucleotide component.

52. The method of claim 51, wherein the composition further comprises a component selected from the group consisting of:
v. a carbohydrate component;
vi. a protein component;
vii. an ω-3 fatty acid component; and
viii. combinations thereof.

53-56. (canceled)

57. The method of claim 51, wherein the nucleotide component comprises an extract of a yeast selected from the group consisting of Saccharomyces cerevisiae and Candida utilis.

58-60. (canceled)

61. The method of claim 51, wherein the athlete is an endurance athlete.

62. The method of claim 51, wherein the athlete is a strength athlete.

63-74. (canceled)
